(19) United States
(12) Patent Application Publication
(10) Pub. No.: US 2023/0355880 A1
(43) Pub. Date: Nov. 9, 2023

(54) APPLICATOR TUBE

(71) Applicant: Ferrosan Medical Devices A/S, Søborg (DK)

(72) Inventors: Peter Lund Hammershøj, Brønshøj (DK); Henning Uzoma Igwebuike, Lynge (DK)

(21) Appl. No.: 17/780,673

(22) PCT Filed: Dec. 22, 2020
(86) PCT No.: PCT/EP2020/087599

§ 371 Date: May 27, 2022

(30) Foreign Application Priority Data
Dec. 23, 2019 (EP) 19219250.8

(51) Int. Cl. A61M 5/20

(52) U.S. Cl. CPC A61M 5/2046; A61M 2005/3128

(57) ABSTRACT

The invention regards an applicator tube for delivering a paste from a syringe, comprising: a delivery tube comprising a distal end for delivering the paste, and a valve system attached to a proximal end of the delivery tube and configured for attachment to the syringe, the valve system having a first configuration allowing aspiration of gas from the surroundings, and a second configuration allowing expression of aspirated gas into the delivery tube, such that the applicator tube is configured for transporting a volume of aspirated gas into and through the delivery tube when an attached syringe is aspirated and expressed.

### TECHNICAL FIELD

The present invention relates to an applicator tube and a kit of parts for delivering a viscous fluid, such as a paste, more particularly an endoscopic and/or laparoscopic applicator tube for delivering a viscous fluid, such as a paste, as well as a method of emptying the applicator tube, such as the endoscopic and/or laparoscopic applicator tube. Advantageously, the applicator is used for delivering a viscous fluid within an insufflated body part.

### BACKGROUND

A viscous fluid, such as a paste, may be precisely applied to a target site by use of a syringe. A syringe comprises a plunger, or piston, fitted to a barrel with an opening, where the barrel comprises the paste. By pushing or translating the syringe plunger along the barrel, the paste, typical in the form of an essentially non-compressible thick viscous composition, is discharged from the opening of the syringe barrel in a controlled manner. Thus, a paste may be delivered to a target site with high spatial precision and in a flexible dosage, by use of a syringe.

The delivery of a paste to a specific target site and in a precise amount, is especially important for pastes, which are applied for medical purposes, such as for surgical applications. For example, haemostatic compositions for surgical applications are typically in the form of a paste.

An example of an effective surgical haemostat is a gelatine paste comprising a haemostatically effective amount of thrombin. Thrombin is a clotting agent, and may thus be used to control the bleeding at a haemorrhaging site. However, for the medical paste to be haemostatically efficient, it is important that an effective concentration of the thrombin is present in the paste, and that the thrombin is uniformly distributed in the paste, and that the paste has a suitable viscosity and rheology for precise and fixed positioning.

For endoscopic and/or laparoscopic procedures, the target site is not directly accessible for a syringe. Instead the paste is applied from the syringe via an applicator tube, where the applicator tube may be introduced into the body via a trocar port.

Hence, the syringe facilitates a precise amount of paste discharged, and the applicator tube placed within the trocar and associated obturator allows for precise application of the paste to a distal target site.

Discharge or dosage of paste from the applicator tube at the distal target site implicitly means that the tube is filled with a residual or remaining amount of paste. If the residual paste is not subsequently discharged and e.g. applied at a target site, the paste is wasted. Furthermore, the residual paste within the tube may be subject to phase changes, such as hardening of the paste, and when occurring within the tube, this may lead to mechanical stresses and damage of the tube.

To ensure utilization of the residual paste and to avoid damage of the devices, paste application typically implies two steps: 1) application of paste by depressing the plunger of the paste containing syringe, and 2) the residual paste in the applicator tube is discharged by use of a ram rod or stylus.

The second process step typically requires the use of two hands, which is especially challenging in laparoscopic procedures. US 2018 303531 discloses a hemostatic delivery tube, where the residual paste is discharged via a stylus advancing through the tube. When the stylus extends through the entire tube, any remaining paste is avoided.

### SUMMARY

Surgical procedures are typically subject to time constraints, and the time consumption of each medical procedure thus of importance. For example, the time consumption may be critical when using a haemostatic paste for inhibiting bleedings as the surgeon will have to interrupt his procedure while waiting for the applicator with the haemostat to be prepared. Thus, the preparation time of the applicator may cause increased blood loss and longer operating time of the surgical procedure.

For more efficient paste application procedures, improved applicator tubes are needed.

A first aspect of the invention relates to an applicator tube for delivering a paste from a syringe, comprising:
a delivery tube comprising a distal end for delivering the paste,
a valve system attached to a proximal end of the delivery tube and configured for attachment to the syringe, the valve system having a first configuration allowing aspiration of gas from the tube surroundings, and a second configuration allowing expression of aspirated gas into the delivery tube,
such that the applicator tube is configured for transporting a volume of aspirated gas into and through the delivery tube when an attached syringe is aspirated and expressed.

A second aspect of the invention relates to an endoscopic and/or laparoscopic applicator tube for delivering a paste from a syringe, comprising:
a delivery tube comprising a distal end for delivering the paste,
a valve system attached to a proximal end of the delivery tube and configured for attachment to the syringe, the valve system having a first configuration allowing aspiration of gas from the tube surroundings, and a second configuration allowing expression of aspirated gas into the delivery tube,
such that the applicator tube is configured for transporting a volume of aspirated gas into the delivery tube when an attached syringe is aspirated and expressed.

A third aspect of the invention relates to a method of emptying an applicator tube, comprising the steps of:
a) providing the applicator tube according to the first or second aspect,
b) attaching a syringe to the delivery tube,
c) aspirating gas into the syringe,
d) expressing the gas from the syringe into the delivery tube, whereby the applicator tube is emptied.

A fourth aspect of the invention relates to a kit of parts, comprising: a delivery tube, and a valve system, wherein the valve system is configured to be detachably attached to a proximal end of the delivery tube, and further configured for attachment to a syringe, the valve system having a first configuration allowing aspiration of gas from the surroundings, and a second configuration allowing expression of aspirated gas into the delivery tube.

In a preferred embodiment, the kit of parts is for an applicator tube according to the first aspect of the invention.

The present disclosure provides an improved applicator tube, which facilitates a faster, more simple and more efficient paste application, including emptying the applicator tube, where the amount of wasted paste is reduced. Particularly, the present applicator tube facilitates emptying of the applicator from residual paste without the use of separate additional parts, such as a stylus, and without the need of the additional steps associated with introducing a separate part, such as a stylus, because of a valve system attached to the delivery tube of the applicator tube. The emptying of the applicator tube may alternatively, or additionally, facilitate that the applicator tube can be easily reused or recycled. After use, the residual paste may be easily discharged as waste, and the applicator tube immediately reused with a different paste.

In a preferred embodiment, the valve system comprises at least one one-way valve.

More specifically, the present applicator tube facilitates emptying of the applicator from residual paste immediately after discharging paste from the tube delivered from a syringe, and without disconnecting the syringe delivering the paste, because of a valve system attached to a proximal end of the delivery tube and configured for attachment to the syringe, the valve system having a first configuration allowing aspiration of gas, such as air, from the surroundings, and a second configuration allowing expression of aspirated gas, such as air, into the delivery tube, such that the applicator tube is configured for transporting a volume of aspirated gas, such as air, into the delivery tube when an attached syringe is aspirated and expressed.

To reduce the risk of injecting detrimental gasses and detrimental amounts of gasses into the body, the aspirated gas is advantageously a gas that is sufficiently soluble in bodily fluids, such as blood. For example, injection of large amounts of air into a body cavity may cause air embolisms. Hence, advantageously, the gas is aspirated from a gas container comprising a well defined composition, and located in the tube surroundings. Alternatively, or additionally, the gas is aspirated from the tube surroundings placed within the body. For endoscopic and/or laparoscopic procedures, the distal part of the tube is typically positioned within an insufflated body part, meaning that the cavity of the body part has been filled with gas to inflate the cavity to obtain more workroom during the laparoscopic procedure. Examples of typical insufflation gases are air, CO₂, nitrous oxide (N₂O), helium (He).

In a preferred embodiment of the disclosure, the aspirated gas is air from the tube surroundings, and/or an insufflation gas selected from group of: air, CO₂, nitrous oxide (N₂O), helium (He), and combinations thereof.

### DESCRIPTION OF DRAWINGS

The invention will in the following be described in greater detail with reference to the accompanying drawings.

FIG. 1 shows a cross-sectional view of an embodiment of the applicator tube according to the present disclosure.

FIG. 2 shows a perspective view of an embodiment of the applicator tube according to the present disclosure.

FIG. 3 shows a cross-sectional view of an embodiment of the proximal end of an applicator tube according to the present disclosure.

FIG. 4 shows a cross-sectional view of another embodiment of the proximal end of an applicator tube according to the present disclosure.

FIG. 5 shows an embodiment of a process for emptying the applicator tube according to the present disclosure.

FIG. 6 shows a perspective view of an embodiment of the applicator tube, where the enhanced sections shown in circles, shows respectively the attachment to the syringe, and embodiments of the position of the valves within the valve system.

FIG. 7 shows an embodiment of an applicator tube according to the present disclosure placed within an insufflated body part, where gas is either aspirated from the proximal tube surroundings (A), or insufflation gas is aspirated from the distal tube surroundings (B).

FIG. 8 shows an embodiment of an applicator tube according to the present disclosure placed within an insufflated body part, where insufflation gas is aspirated from the distal tube surrounding (A), a close-up of the embodied distal tube section (B), and a cross-sectional view of a tube section (C).

FIG. 9 shows an embodiment of a valve system according to the present disclosure, in the form of a two valve function combination valve, such as a duckbill/umbrella combination valve. (A) The valve may be integrated into the proximal end of the delivery tube, such as integrated into the transition unit, and the inner duckbill valve may be either closed, as seen to the left in (B), or open for flow in the direction of the pointed beak, as shown by arrow to the right in (B). Correspondingly, the outer umbrella valve may be either closed, as seen to the right in (C), or open for flow in the direction of the inverted umbrella, as shown by arrow to the right in (C).

FIG. 10 shows embodiments of the applicator tube lumens according to the present disclosure, where (A) and (B) show perspective views of the distal end of the applicator tube, and (C-G) show cross-sectional views of the lumen configuration, where the second lumens may be placed within the delivery tube wall (C), or on the outside of the delivery tube wall (D, F, G), or inside the delivery tube wall (E).

### DETAILED DESCRIPTION

The invention is described below with the help of the accompanying figures. It would be appreciated by the people skilled in the art that the same feature or component of the device are referred with the same reference numeral in different figures. A list of the reference numbers can be found at the end of the detailed description section.

Applicator Tube

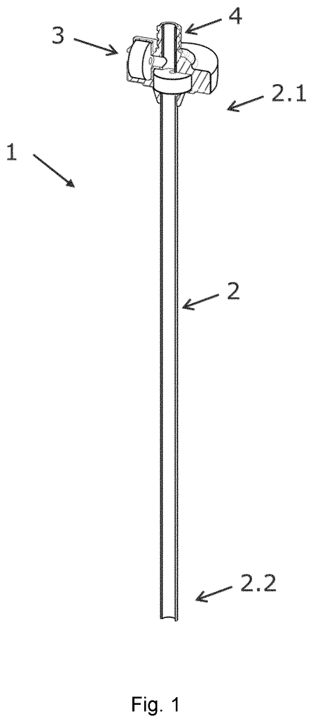

FIG. 1 shows an embodiment of an applicator tube 1 according to the present disclosure, comprising a delivery tube 2 with a proximal end 2.1 and a distal end 2.2, where the proximal end is adapted for forming a connection to a syringe. When a syringe containing a paste is attached to the proximal end, said paste may be delivered, dispensed or discharged from the distal end of the delivery tube by pushing the plunger, whereby the paste is first transferred from the syringe into the delivery tube, and from the delivery tube expelled through it's distal end.

The applicator tube is advantageously applied for endoscopic and/or laparoscopic surgical procedures, where the delivery tube is introduced into the internal body via the hollow tube, or cannula, of a trocar. Typically, a trocar is placed through the abdomen during the laparoscopic surgery, and subsequently used as a portal for the following procedures. Thus, the endoscopic and/or laparoscopic applicator tube advantageously have a length, diameter and stiffness compatible with and enabling easy manipulation and precise positioning of the applicator tube within a trocar. Particularly, the applicator tube must have a length, diameter, and stiffness or hardness allowing manipulation of the distal end of the applicator tube via the proximal end, which is accessible to the user located at the trocar port.

In an embodiment of the disclosure, the applicator tube is an endoscopic and/or laparoscopic applicator tube. In a further embodiment, the applicator tube is adapted for insertion into a trocar. In a further embodiment, the applicator tube has a length between 20-150 cm, more preferably between 25-80 cm, such as between 30-60 cm. In a further embodiment, the applicator tube comprises a tube with an internal diameter of between 2-15 mm, more preferably between 3-8 mm, such as between 4-6 mm or 3-5 mm. In a further embodiment, the applicator tube comprises a tube containing a volume of between 3-20 ml, preferably between 4-10 ml or 5-10 ml, such as 5 ml.

In a further embodiment, the applicator tube comprises a tube with a stiffness of equal to or above 0.5, 1.5 or 2 GPa as measured by tensile test according to the appropriate standard EN10002, e.g. EN10002-1 (ISO 6892-1) as standard for metal stiffness/tensile E modulus, and/or ISO 527-1/-2, ISO 527-4, ISO 527-5, ASTM D638 as standard for the tensile E-modulus for plastics, polymers, composite material, and/or ISO 178/ASTM D790 as standard for the flexural E-modulus for plastics, polymers, composite material. More preferably, the applicator tube comprises a tube with a stiffness above 50 or 60 GPa. Examples of materials with a stiffness above 0.5 or 1.5 GPa include plastics, metals, polymers, glass, glass fibers, carbon fibers, polymer fibers, composites such as fiber-reinforced materials and combinations thereof. In an embodiment of the disclosure, the applicator tube comprises a tube consisting of a material selected from the group of: metals, plastics, polymers, glass, glass fibers, carbon fibers, polymer fibers, composites such as fiber-reinforced materials and combinations thereof.

In a further embodiment, the applicator tube comprises a tube with a stiffness below 0.5 GPa. Such materials have in addition, or alternatively, a preferred hardness. An example of a material with a stiffness below 0.5 GPa is a thermoplastic elastomer with a Shore Durometer A and/or D hardness according to standard ISO 868/ASTM D2240.

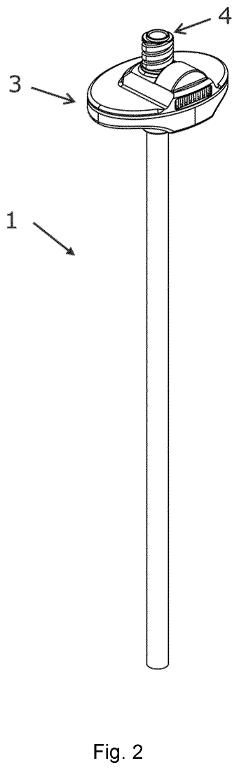

For handle ergonomics and for facilitating precise manipulation and positioning of the applicator tube within the trocar, the applicator tube advantageously comprises a handle or a grip attached to the proximal end of the delivery tube. To improve the compactness and robustness of the applicator tube, the handle is advantageously in the form of a transition unit between the delivery tube and the syringe, such that the syringe is attachable to the transition unit. FIGS. 1-2 show embodiments of an applicator tube comprising a transition unit 3, which may further act as a handle.

In an embodiment of the disclosure, the applicator tube comprises a transition unit attached to the proximal end of the delivery tube, wherein preferably the transition unit is adapted as a handle.

To ensure easy attachment of the syringe to the delivery tube, and further to ensure easy and safe transfer of the paste from the syringe into the delivery tube, the syringe and delivery tube are advantageously detachably attachable. An example of a detachable attachment is a Luer fitting or Luer lock, where a male-taper fitting of a first component is connected to a mating female part of a second component. A Luer fitting further has the advantage of providing an essentially leak-free connection between the two components. Hence, the applicator tube advantageously comprises a Luer lock 4 for attaching the syringe, placed at the proximal end adapted for attaching the syringe, as exemplified in FIGS. 1-2. For example, the Luer lock may be placed at the proximal end of the delivery tube or at the transition unit. The Luer lock may be according to ISO 80369-7.

In an embodiment of the disclosure, the applicator tube comprises a Luer lock for attaching a syringe. In a further embodiment, the delivery tube and/or the transition unit comprises a Luer lock for attaching a syringe.

The attachable syringe is advantageously pre-filled with paste before being attached to the delivery tube. Alternatively, an empty syringe may be attached to the delivery tube, where the empty syringe is further configured for being filled with paste in the same manner as a cartridge by removing the plunger, while in the attached configuration. Further alternatively, an empty syringe, optionally pre-filled with air, may be attached to the delivery tube to expel air, as described below.

In an embodiment of the disclosure, the applicator tube is adapted for attaching a pre-filled syringe, such as a syringe pre-filled with paste. In another embodiment, the applicator tube is adapted for attaching an empty syringe.

It follows that the presently disclosed applicator tube for delivering a paste from a syringe may be used with any type of syringe. Examples of syringes include single chamber syringes and syringes comprising multiple chamber, such as dual chamber syringes, where the contents of the multiple chambers may be mixed prior to injection.

To facilitate easy paste delivery from the applicator tube, the plunger of the syringe is adapted to be pushable by use of a user's hand or thump. To ensure paste delivery by application of moderate hand pressure, the applicator tube or delivery tube is adapted to have a sufficiently large internal diameter at the distal end discharging the paste. To further facilitate precise delivery of the paste at a target site, the applicator tube or delivery tube advantageously have a sufficiently small internal diameter at the distal end. Easy and precise delivery of a paste from a syringe and applicator tube may be obtained by an applicator tube or delivery tube comprising a cannula. Further advantageously, the delivery tube is a cannula, where by the term “cannula” is meant a tube that can be inserted into the body. For example it may be a tube, which concludes with a spike/angular open end to provide fluid access through the entire cannula.

In an embodiment of the disclosure, the delivery tube comprises or is a cannula.

Formable Distal Section

To ensure easy manipulation, as well as precise and flexible paste delivery, the applicator tube advantageously comprises a formable section. Optionally, the entire applicator tube is formable. Advantageously, the formable section is at least a distal section of the tube, such as a formable distal tip of the delivery tube. Thus, the tube may be formed or shaped into a desired shape or configuration by applying a deformation force, typically by hand, to bend the formable section. The tube will then retain the configuration until a further deformation force is applied to form the tube into a different configuration.

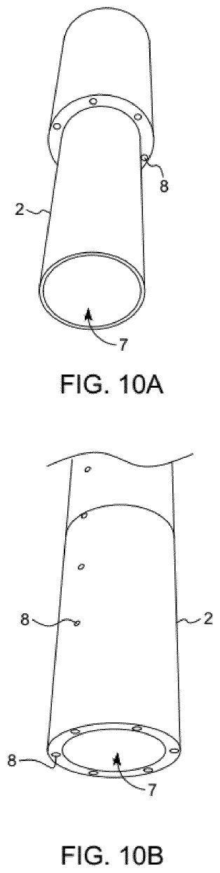

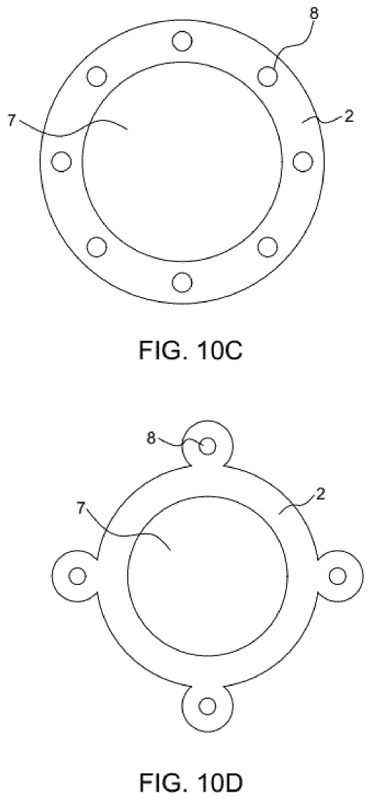

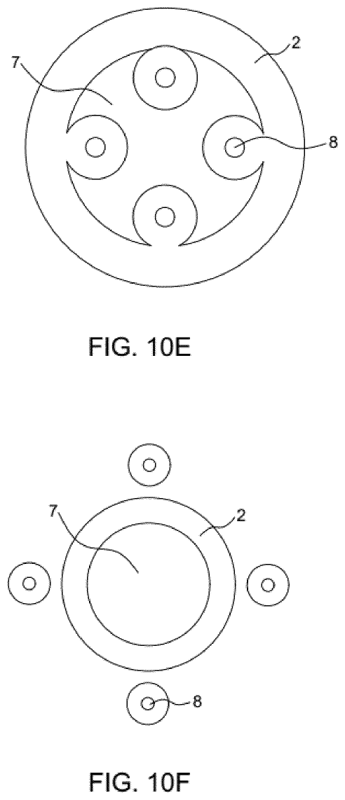

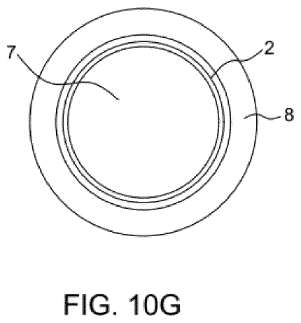

The formable section may be obtained by the formable section comprising a malleable member, as described in WO 2011/047753. The malleable member is made of a suitable material, which is configured to maintain a configuration after a deformation. For example, the malleable member may be a metal, e.g. comprising aluminum or an aluminum alloy, and be in the shape of a wire or mesh, which thereby may be formed/deformed into a desired shape, preferably by manual bending. The malleable member is integrated into the applicator tube or the delivery tube, e.g. the malleable member may be received within a lumen of the applicator tube, thereby forming a formable section. Examples of tube lumens, wherein a malleable member in the form of a wire or a mesh lumen may be integrated are shown in FIG. 10. The delivery tube comprises a first lumen 7 for discharging the paste, and at least one of the surrounding second lumens 8 may receive a malleable member. For example, a malleable wire may be located within a lumen within the delivery tube wall, as shown in FIG. 100, or located in a lumen placed on the outside or inside of the delivery tube wall (FIG. 10D-G). Similarly, a malleable mesh may be rolled into a malleable cylinder, which may be located within any of the lumens of FIG. 10. Alternatively, a malleable mesh may be located in a lumen, which is concentric with the delivery tube opening, as shown in FIG. 10G.

In an embodiment of the disclosure, the applicator tube comprises a formable distal section configured to be shaped into a desired configuration. In a further embodiment, the formable distal section comprises a malleable member configured to maintain a configuration after a deformation, wherein said malleable member optionally is a malleable wire or a malleable mesh. In a further embodiment, the malleable member is located on the inside, outside, or within the delivery tube wall.

Applicator Tube Emptying

When the applicator tube has delivered paste from the syringe, the applicator tube including the entire length of the delivery tube, will be filled with remaining or residual paste. To utilize the residual paste, e.g. to apply it to the first target site or a second target site, the applicator tube must be emptied. According to the present disclosure, the applicator tube and particularly the delivery tube, may be emptied by a gas pressure, such as an air pressure. The embodiments of the disclosure may be extended to any type of gas, but will in the following be exemplified based on air. By the term emptying is meant removal or cleaning of paste from the applicator tube. More specifically the term emptying means removal of residual paste, i.e. the paste remaining in a tube following a paste discharge from the tube.

Alternatively, or additionally, the applicator tube may be emptied to facilitate simple, easy, and controlled reuse or recycling of the applicator tube. After use, the residual paste may be discharged and disposed as waste, and the applicator tube immediately reused with a different paste.

The gas/air pressure required for discharging the residual paste will depend on factors such as the internal cross-sectional area of the tube, the length of the tube, the stiffness of the tube, the paste viscosity, and the wetting properties between the paste and the tube material. For easy emptying of the applicator tube, and particularly the delivery tube, the delivery tube advantageously have a length, diameter, stiffness, and other material properties, which facilitate a produced flow and thus the emptying process.

In an embodiment of the disclosure, the delivery tube has a length between 20-150 cm, more preferably between 25-80 cm, such as between 30-60 cm. In a further embodiment, the delivery tube comprises an internal diameter of between 2-15 mm, more preferably between 3-8 mm, such as between 4-6 mm or 3-5 mm. In a further embodiment, the delivery tube comprises a tube containing a volume of between 3-20 ml, preferably between 4-10 ml, such as 5 ml. In a further embodiment, the delivery tube has a stiffness of equal to or above 0.5, 1.5 or 2 GPa as measured by the appropriate standard, e.g. tensile test according to the standard EN10002, or EN10002-1 (ISO 6892-1) as standard for metal stiffness/tensile E modulus, and/or ISO 527-1/-2, ISO 527-4, ISO 527-5, ASTM D638 as standard for the tensile E-modulus for plastics, polymers, composite material, and/or ISO 178/ASTM D790 as standard for the flexural E-modulus for plastics, polymers. More preferably, the delivery tube has a stiffness above 50 or 60 GPa. In an embodiment of the disclosure, the delivery tube comprises a material selected from the group of: metals, plastics, polymers, glass, glass fibers, carbon fibers, polymer fibers, composites such as fiber-reinforced materials, and combinations thereof.

For simple and fast emptying, the applicator tube is advantageously configured such that the residual paste may be removed and discharged from the distal end of the delivery tube by an air/gas pressure generated by a syringe. For example, a syringe may be pre-filled with air/gas, or aspirated to store air/gas, and then attached to the proximal end of the delivery tube. Upon injecting or expressing the syringe stored gas/air into the delivery tube, the generated air pressure causes the residual paste to be discharged from the delivery tube.

Emptying the applicator tube by use of the moderate gas/air pressure produced by a syringe operated by hand further has the advantage that the discharge of paste from the delivery tube may be precisely controlled. Advantageously, the rate of advancing the piston of the syringe, corresponds to the rate of paste discharge from the tube. Hence, precise and controlled paste delivery to a target site may be obtained.

A more efficient emptying procedure may be obtained if the gas/air pressure is generated by an already attached syringe. This means that the gas/air pressure is generated without disconnecting the syringe from the applicator tube. Thus, the steps of disconnecting the syringe containing paste, and subsequent attachment of a syringe containing gas/air is avoided, and a faster and more efficient removal of the residual paste is obtained. Emptying by use of a syringe attached to the applicator tube may be obtained by the valve system according to the present disclosure.

In an embodiment of the disclosure, a method of emptying the applicator tube comprises the steps of:
a) providing an applicator tube according to the present disclosure,
b) attaching a syringe to the delivery tube,
c) aspirating gas into the syringe,
d) expressing the gas from the syringe into the delivery tube, whereby the applicator tube is emptied.

To further improve the efficiency of the process, the attached syringe may be the syringe containing the paste. Thus, when the applicator tube has delivered the desired amount of paste from the syringe, the delivery tube is emptied from residual paste by aspirating gas/air into the syringe without disconnecting the syringe, and subsequently expressing the aspirated into the delivery tube.

Preferably, the entire amount of paste contained in the syringe is expressed before aspirating gas/air. Hence preferably, the applicator tube is emptied from residual paste by first expressing any remaining paste from the syringe into the delivery tube, and then aspirating gas/air into the syringe without disconnecting the syringe. The gas/air aspirated into the syringe void of paste, is then expressed into the applicator tube and particularly the delivery tube.

In an embodiment of the disclosure, the attached syringe in (b) contains paste, and the method further comprises the step of expressing at least a part of the paste from the syringe into the delivery tube before aspirating gas/air in (c).

It follows that the volume of gas/air aspirated from the surroundings in step (c) is limited by the size of the syringe. Hence, the gas/air pressure generated by the syringe is determined by the size of the syringe and the force pushing the plunger. To facilitate simple and efficient emptying, the volume of gas/air aspirated from the surroundings in step (c) is preferably between 4-100 ml, more preferably between 5-20 ml, such as between 10-15 ml or between 5-10 ml.

In an embodiment of the disclosure, the applicator tube is adapted for aspirating a volume of gas/air from the surrounding of between 4-100 mL, more preferably between 5-20 ml, such as between 10-15 ml or between 5-10 ml.

Additional volumes of gas/air may be aspirated from the surroundings by repeating steps (c) and (d). For example the steps may be repeated at least 2, 3, 4, 5, or 6 times.

In an embodiment of the disclosure, the steps (c) and (d) are repeated.

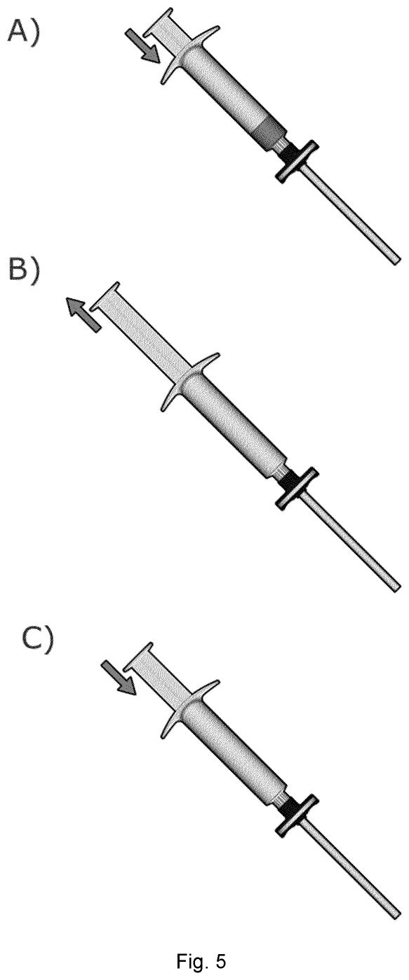

FIG. 5 shows an embodiment of a process for emptying the applicator tube according to the present disclosure, where the movement of the plunger is indicated by arrow. FIG. 5A shows the first step, where the paste, e.g. a gelatin paste, is expressed until the syringe is empty. FIG. 5B shows the second step, where the plunger of the syringe, while still attached to the applicator tube, is pulled back or aspirated, to fill the syringe with gas/air. FIG. 5C shows the third step, where plunger is injected such that the aspirated volume of gas/air generates an air/gas pressure into the applicator tube, such that the applicator is emptied from the gelatin paste.

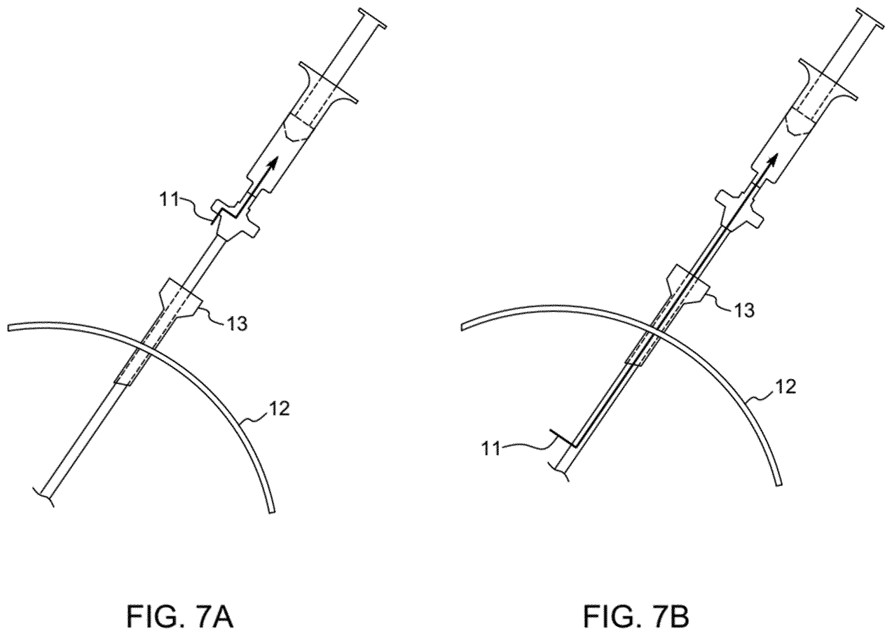

FIG. 7 shows an embodiment of an applicator tube according to the present disclosure placed within an insufflated body part or body cavity 12, e.g. an insufflated stomach. The insufflated body part is accessed via a trocar 13, such that the distal end of the delivery tube is located within the insufflated body part, and a syringe including a paste is attached to the proximal end of the delivery tube. After expressing the paste from the syringe, the syringe plunger may be retracted, and gas 11 from the surroundings may be aspirated into the syringe without removing the syringe or the applicator. This is obtained via the valve system described further below, and the gas may be aspirated either from the proximal tube surroundings as seen in FIG. 7A, or aspirated from the distal tube surroundings, i.e. in the form of insufflation gas, as seen in FIG. 7B.

In an embodiment of the disclosure, the gas is atmospheric air from the surroundings. In another embodiment of the disclosure, the gas is an insufflation gas selected from group of: air, CO₂, nitrous oxide (N₂O), helium (He), and combinations thereof.

Valve System

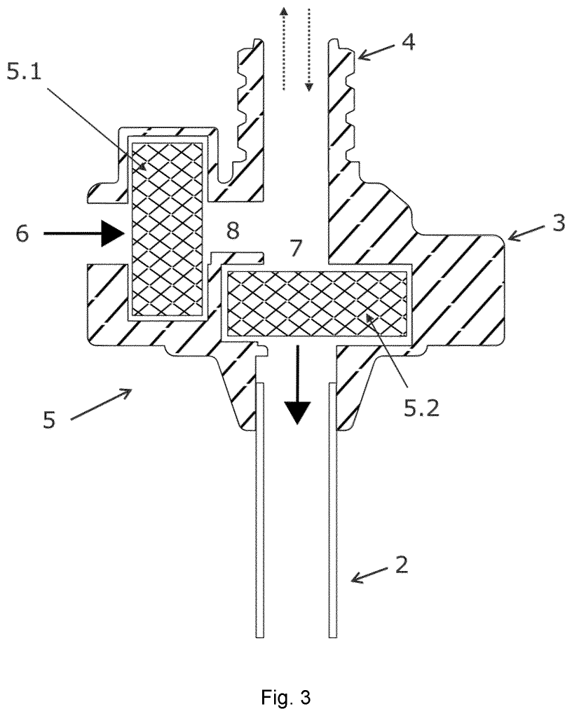

FIG. 3 shows an embodiment of the valve system 5 according to the present disclosure, which is attached to the proximal end of the delivery tube 2.

For a compact and robust applicator tube, the valve system is advantageously integrated into the transition unit or handle 3, and the valve system is further advantageously configured for attachment to the syringe, e.g. via a Luer lock 4, as illustrated in FIG. 3. However, to reduce the number of parts, the skilled person will know that the valve system may also be directly attached to the proximal end of the delivery tube.

In an embodiment of the disclosure, the valve system is integrated into the transition unit, and/or the proximal end of the delivery tube.

The valve system is configured to have two configurations: a first configuration allowing aspiration of gas/air from the surroundings, and a second configuration allowing expression of aspirated gas/air into the delivery tube. This implies that the applicator tube is configured for transporting a volume of aspirated gas/air into the delivery tube, when an attached syringe is aspirated and expressed.

The skilled person knows that such controlled fluid flows may be obtained by use of a valve system, comprising one or more valves, where a valve is defined as a device that regulates, directs or controls the flow of a fluids (i.e. gases, liquids, and fluidized solids, such as paste and slurries) by opening, closing, and/or partially obstructing the flow passageway. Thus, an example of a valve includes a flow constriction element, such as a protrusion within a fluid passageway, where the protrusion blocks fluid passage, when the fluid pressure is below a threshold value, and when the fluid pressure is above the threshold valude, the fluid flows and circumvents the protrusion. A valve including a flow constriction element is also referred to as a “constriction valve”.

A valve may further be adapted to regulate, direct or control the flow of specific fluids. For example, a valve may be adapted to regulate the flow of paste, whereas the flow of the gaseous phases are not affected by the valve. An example of a valve adapted to regulate the flow of specific fluids is a constriction valve, where the dimension of the constriction element is configured to allow the flow of gas in both directions, but only allow flow of paste of a certain viscosity in one direction. Hence, a constriction valve may be adapted to be a two-way valve for the flow of gas, and a one-way valve for the flow of paste. For example, a constriction valve may allow the flow of paste from the syringe into the dispensing tube, but the paste is not allowed to passage the constriction in the opposite direction.

A valve may further be a one-way valve or check valve, meaning that the valve only allows the fluid to flow in one direction. Hence, a one-way valve has two positions, an “open” and “closed” position, where in the open position the valve provides fluid passageway in one direction, and in the closed position provides no fluid passageway.

The opening/closing of a one-way valve may be operated in response to magnetic forces, gravity, and/or fluid pressure. For example, a one-way valve may open in response to a fluid pressure exceeded a predefined threshold value. A valve may further be operated as a one-way valve by being adapted to have a regulated and controllable flow direction.

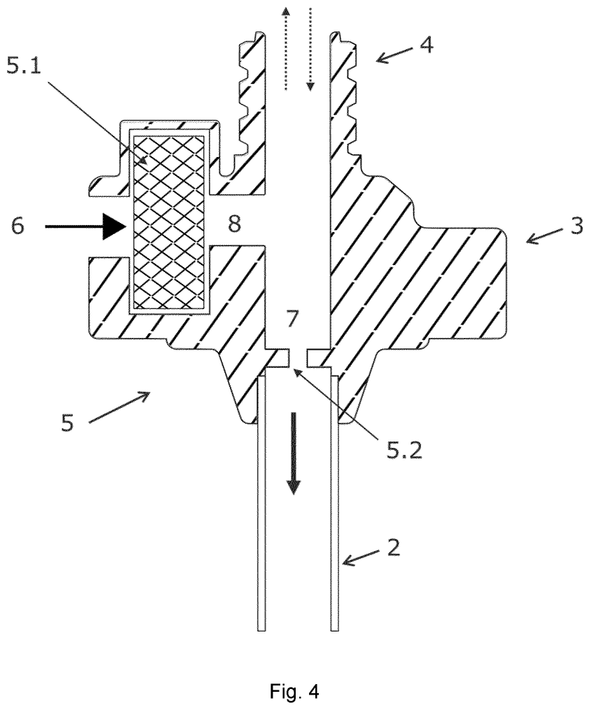

In an embodiment of the disclosure, the valve system comprises at least two valves 5.1 and 5.2 as illustrated in FIG. 3. The first valve is a one-way valve or a valve adapted to have a controllable flow direction, and the second valve is also a one-way valve or a valve adapted to have a controllable flow direction. For example, both the first valve and the second valve may be one-way valves, as exemplified in FIG. 3. Alternatively, the second valve may be a valve adapted to have a controllable flow direction, as exemplified in FIG. 4, where the second valve is in the form of a flow constriction element.

FIG. 3 shows an embodiment of the valve system comprising two one-way valves 5.1 and 5.2. The valve system has two configurations: a first configuration allowing aspiration of air from the surroundings 6 and through the first one-way valve 5.1 and the second lumen 8, as indicated by solid arrow and the dotted arrow to the left in FIG. 3, and a second configuration allowing expression of the aspirated air through the second one-way valve 5.2 and the first lumen 7, and into the delivery tube 2, as indicated by solid arrow and the dotted arrow to the right in FIG. 3.

A syringe attached to the proximal end of the delivery tube, e.g. at the Luer lock illustrated in FIG. 3, may be aspirated while in the attached position, whereby gas/air is aspirated from the surroundings, through the first one-way valve, and further transferred into the syringe, where it may be stored in the barrel. Due to the valve system, fluid communication is established only between the surroundings and the attached syringe, and the aspirated gas/air will bypass the delivery tube. The stored gas/air may subsequently be expressed by pushing the plunger, and due to the valve system, the volume of aspirated gas/air will only flow through the second one-way valve and into the delivery tube. Hence, the applicator tube is configured for transporting a volume of aspirated gas/air into the delivery tube, when an attached syringe is aspirated and expressed. Example 1 further describes an embodiment of the applicator tube configured for use with a syringe comprising a low-viscosity paste.

FIG. 4 shows an embodiment of the valve system comprising a first one-way valve 5.1 and a second valve 5.2 in the form of a flow constriction element or a constriction valve. Similar to FIG. 3, the valve system has two configurations: a first configuration allowing aspiration of gas/air from the surroundings 6 and through the first one-way valve 5.1 and the second lumen 8, as indicated by solid arrow and the dotted arrow to the left in FIG. 4, and a second configuration allowing expression of the aspirated gas/air through the second valve 5.2 and the first lumen 7, and into the delivery tube 2, as indicated by solid arrow and the dotted arrow to the right in FIG. 4.

A syringe attached to the proximal end of the delivery tube, e.g. at the Luer lock illustrated in FIG. 4, may be aspirated while in the attached position, whereby gas/air is aspirated from the surroundings, through the first one-way valve, and further transferred into the syringe, where it may be stored in the barrel. Due to the valve system, fluid communication is established essentially only between the surroundings and the attached syringe, and the aspirated gas/air will bypass the delivery tube. Also, restricted fluid communication may be established between the delivery tube and the attached syringe, depending on the dimensions of the constriction element and the content of the delivery tube. Particularly, if the constriction element reduces the cross sectional area of the delivery tube with between 20-90%, and/or the delivery tube contains a high-viscous residual paste, aspiration of residual paste is prevented. The stored gas/air may subsequently be expressed by pushing the plunger, and due to the valve system, the volume of aspirated gas/air will only flow through the second valve and into the delivery tube. Hence, the applicator tube is configured for transporting a volume of aspirated gas/air into the delivery tube, when an attached syringe is aspirated and expressed. Example 2 further describes an embodiment of the applicator tube configured for use with a syringe comprising a high-viscosity paste.

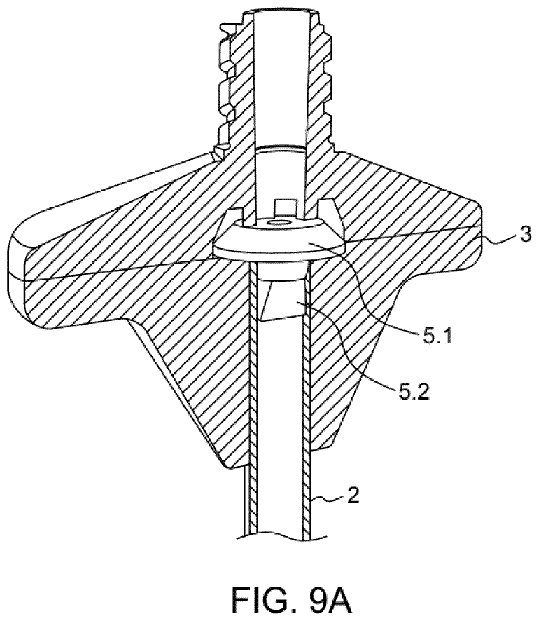

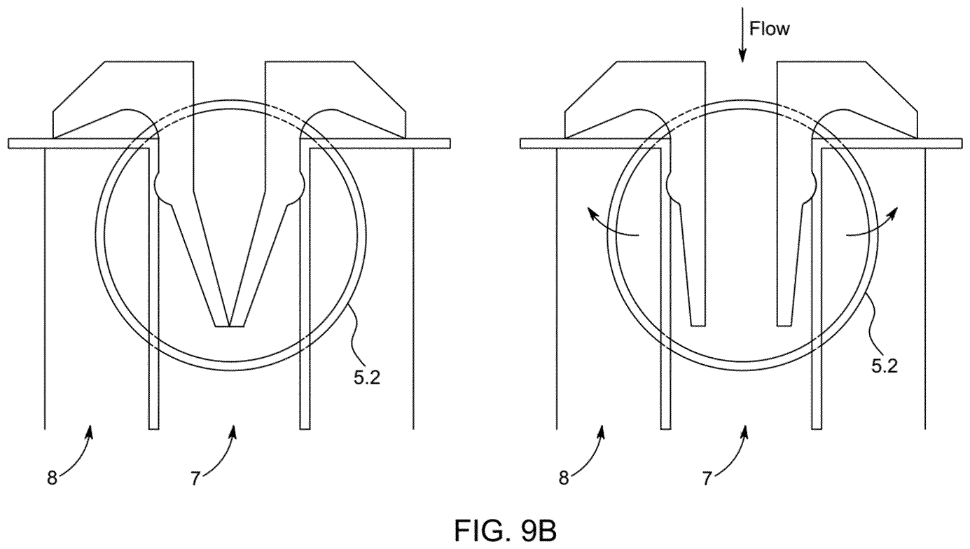

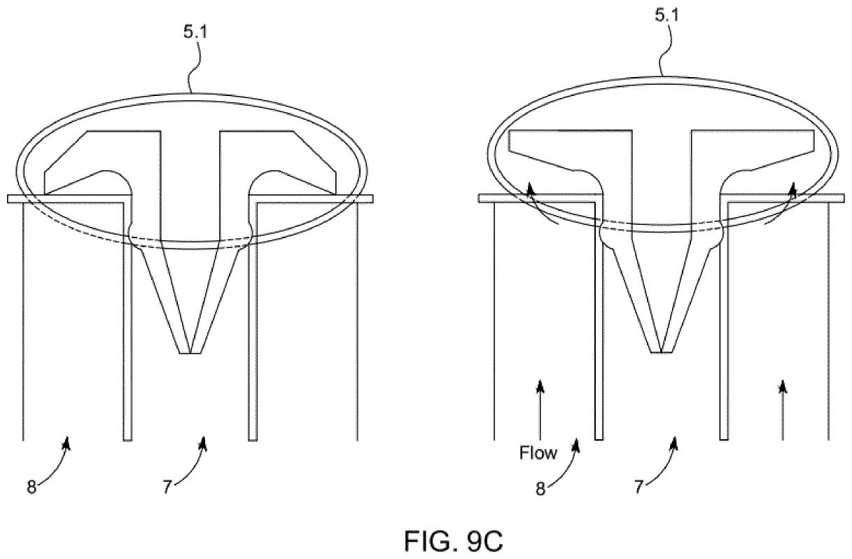

A valve system configured to have two configurations: a first configuration allowing aspiration of gas/air from the surroundings via a second lumen, and a second configuration allowing expression of aspirated gas/air into the delivery tube via a first lumen, may correspondingly be obtained by a two valve function combination valve, such as a duckbill/umbrella combination valve, as shown in FIG. 9.

The combination valve comprises an inner duckbill valve 5.2 having at least one deformable flap, e.g. two rotatable flaps, as seen in FIG. 9B, where the flaps form a sealed connection within a first lumen 7 when exposed to no/low pressure, as seen in FIG. 9B to the left, and where the flaps are separated to form an opening in the seal and the lumen, when exposed to a certain threshold pressure, as seen in FIG. 9B to the right. Hence, the inner duckbill valve acts as a one-way valve for flow in the direction of the beak, as indicated by arrow in the FIG. 9B to the right. The combination valve further comprises an outer umbrella valve 5.1 comprising a deformable flap, which may either form a sealed connection within a second lumen 8, e.g. towards a surface when exposed to no/low pressure, as seen in FIG. 9C to the left, and when exposed to a pressure above a certain threshold, the deformable flap separates from the surface to form an opening, as seen in FIG. 9C to the right. Hence, the umbrella valve also acts as a one-way valve for flow in a direction opposite the beak.

Hence, a syringe attached to the proximal end of the delivery tube, e.g. at the Luer lock illustrated in FIG. 9A, may be aspirated while in the attached position, whereby gas/air is aspirated from the surroundings, through the umbrella valve, and further transferred into the syringe, where it may be stored in the barrel. Due to the valve system, fluid communication is established only between the surroundings and the attached syringe, and the aspirated gas/air will bypass the delivery tube. The stored gas/air may subsequently be expressed by pushing the plunger, and due to the valve system, the volume of aspirated gas/air will only flow through the duckbill valve and into the delivery tube. Hence, the applicator tube is configured for transporting a volume of aspirated gas/air into the delivery tube, when an attached syringe is aspirated and expressed. Example 3 further describes an embodiment of the applicator tube configured for use with a syringe comprising a low-viscosity paste.

In an embodiment of the disclosure, the valve system comprises at least two valves, or a two valve function combination valve, such as a duckbill/umbrella combination valve. In a further embodiment, the valve system comprises at least a first one-way valve, and/or a first constriction valve. In a further embodiment, the valve system comprises at least two one-way valves. In a further embodiment, the valve system comprises a valve with a cross sectional area of between 20-90%, more preferably between 30-80%, and most preferably between 40-60% of the cross sectional area of the delivery tube.

Instead of attaching the valve system directly to the proximal end of the delivery tube, the valve system is advantageously integrated into the transition unit or handle to obtain a compact and robust applicator tube. FIGS. 3-4 also shows embodiments, where the valve system is integrated into the transition unit 3, which is shaped as a circular handle. Also the combination valve may be integrated into the proximal end of the delivery tube, such as integrated into the transition unit, as shown in FIG. 9A.

Lumen

As described above, the valve system or transition unit may include a first lumen 7 and a second lumen 8, where the second lumen is configured for aspirating gas from the surroundings via the first valve 5.1, and where the first lumen is configured for discharging the aspirated gas through the delivery tube, as well as discharging the paste 10, via the second valve 5.2.

More specifically, as illustrated in FIGS. 3-4 and 9, the valve system or transition unit comprises a first and a second lumen, where the first lumen 7 has a first proximal opening and a first distal opening, the first proximal opening corresponding to the attachment to the syringe, and the first distal opening being in fluid communication with the delivery tube, such that the first lumen is configured for discharging the paste and the aspirated gas to a target site. The second lumen 8 has a second proximal opening and a second distal opening, where the second distal opening is in fluid communication with the surroundings, such that the second lumen is configured for aspirating gas from the surroundings at the second distal opening. Optionally, the first proximal opening and the second proximal opening are the same as illustrated in FIGS. 3-4 and 9, such that they correspond to the attachment to the syringe.

In an embodiment of the disclosure, the valve system or the transition unit comprises a first lumen having a first proximal opening and a first distal opening, wherein the first distal opening is in fluid communication with the delivery tube, and at least one second lumen having a second proximal opening and a second distal opening, wherein the second distal opening is in fluid communication with the tube surroundings. In a further embodiment, the first proximal opening and the second proximal opening are the same.

It follows that the second distal opening acts as the entry point for aspirating gas from the surroundings and into the syringe. The second distal opening may be located within a proximal end of the applicator tube or delivery tube, as illustrated in FIG. 7A, whereby gas is consequently aspirated from the proximal tube surroundings, i.e. the atmospheric vicinity of the operator. The second distal opening may also be located within a distal end of the applicator tube or delivery tube, as illustrated in FIG. 7B. Consequently, gas is aspirated from the distal tube surroundings, e.g. the insufflated body part.

FIGS. 3-4 show an embodiment, wherein the second distal opening is located within a proximal end of the applicator tube, such as within the transition unit. This has the advantage of a simple and compact design of the second lumen 8, e.g. the extension of the second lumen may be short and oriented perpendicularly to the first lumen 7, as shown in FIGS. 3-4. Moreover, the second lumen may be in fluid communication with a gas container, which optionally is directly detachably attached to the second distal opening.

In an embodiment of the disclosure, the second distal opening is located within a proximal end of the delivery tube, such as within the transition unit. In a further embodiment, the extension of the second lumen is oriented at an angle from the extension of the first lumen, such as extending perpendicular to the first lumen. In a further embodiment, the second distal opening is in fluid communication with a gas container.

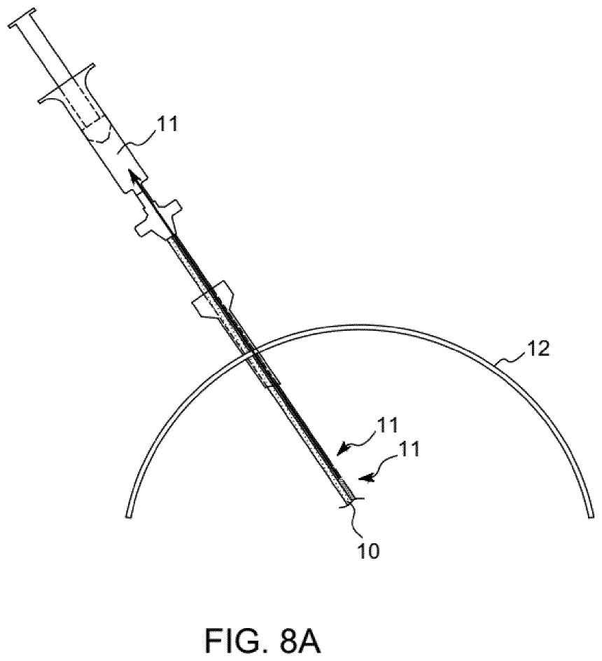

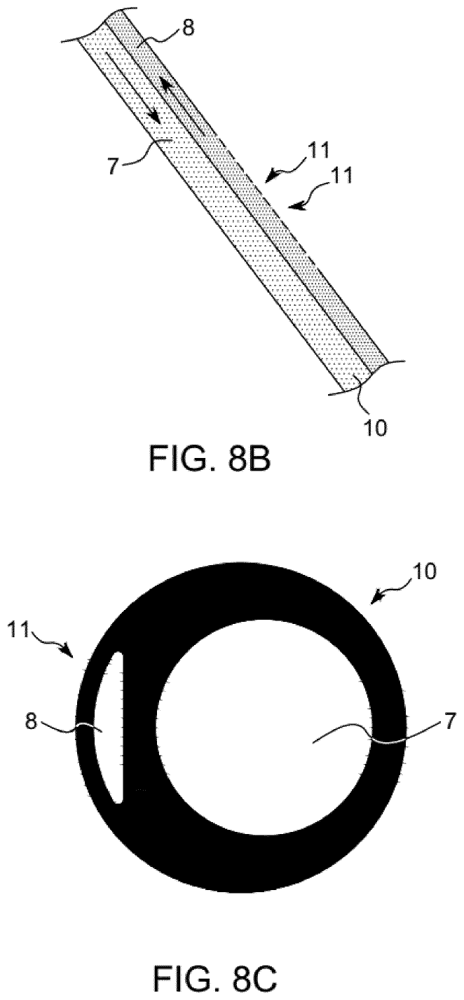

Alternatively, the second distal opening is located within a distal end of the applicator and delivery tube, as illustrated in FIG. 8. This has the advantage that insufflation gas may be used for emptying the applicator tube. In this case, the second lumen 8 for the aspirating gas 11 extends in parallel with the first lumen 7 and the delivery tube, which delivers the paste 10 to the target site, as seen in FIGS. 8A and B. To reduce the risk of aspirating blood or other bodily fluids from the target site, the second distal opening is advantageously located at a distance from the distal end or distal tip of the delivery tube, such as a distance below 15 cm from the distal end.

In an embodiment of the disclosure, the extension of the second lumen is in parallel to the extension of the first lumen. In a further embodiment, the second distal opening is located within a distal end of the delivery tube, optionally at a distance below 2, 5, 6, 7, 8, 10, or 15 cm from the distal end of the delivery tube.

In addition, the risk of aspirating bodily fluids, as well as the force needed for aspirating an insufflated gas, will depend on size and geometry of the second lumen and the distal opening. Advantageously, the size of the second lumen is smaller than the first lumen, as seen in cross-sectional view as seen in FIG. 8C. For example, the cross-sectional size of the second lumen may be dimensioned such that the lumen is located within the delivery tube wall, as exemplified in FIGS. 8C, 10B and 10C. Alternatively, the second lumen may be located on the inside or outside of the delivery tube wall, as exemplified in FIGS. 10D-F, or the second lumen may be concentric with the first lumen, as exemplified in FIG. 10G.

In an embodiment of the disclosure, the second lumen is located on the inside, outside, or within the delivery tube wall. In another, or further embodiment, the second lumen is concentric with the first lumen.

To further reduce the force needed for aspirating an insufflated gas, and to improve the aspiration efficiency, the applicator advantageously comprises multiple second lumens, as illustrated in FIGS. 10A-F. For example, the applicator may comprise eight second lumens, as exemplified in FIGS. 10A and C, six second lumens as in FIG. 10B, or four second lumens as in FIG. 10D-F.

In an embodiment of the disclosure, the applicator tube comprises multiple second lumens, such as 2, 4, 6, 8, or 10 second lumens.

For each second lumen, the second distal opening may advantageously comprise multiple apertures located at different distances from the distal end/tip of the delivery tube, as exemplified in FIG. 8B, such that the insufflated gas may be aspirated at multiple apertures along the tube length. FIG. 10B also shows an embodiment, where multiple apertures are located along the delivery tube, as seen on the outside wall of the tube. In addition, or alternatively, the second lumen may extend to the distal end of the delivery tube, such that the apertures are located at a delivery tube wall flange, either at the tip wall flange, as exemplified in FIG. 10B, or at a distance from the delivery tip, as exemplified in FIG. 10A.

In an embodiment of the disclosure, the second distal opening comprises one or more apertures located at the delivery tube outside wall and/or at the delivery tube wall flange.

FIGS. 3-4 and 9 show embodiments of the valve system integrated into the transition unit, where a first valve is placed within the second lumen and a second valve is placed in the first lumen. To ensure efficient aspiration of gas/air, and restrict the expression of gas/air into the surroundings, the first valve placed in the second lumen is advantageously a one-way valve.

In an embodiment of the disclosure, the valve system comprises at least a first one-way valve. In a further embodiment, the first one-way valve is placed within the second lumen.

Advantageously, the second valve is a second one-way valve placed in the first lumen.

Alternatively, the second valve comprises a part with a reduced cross section area. For example, the second valve advantageously restricts the cross sectional area of the first lumen with between 20-90%.

In an embodiment of the disclosure, the valve system comprises a second one-way valve. In a further embodiment, the second one-way valve is placed within the first lumen.

In another embodiment of the disclosure, the first lumen comprises a part with a reduced cross section area. In a further embodiment, the first lumen comprises a part with a reduced cross sectional are of between between 20-90%, more preferably between 30-80%, and most preferably between 40-60% of the cross sectional area of the lumen.

It follows from FIG. 4 that the second valve 5.2 in the form of a constriction valve may be placed at any position along the longitudinal first lumen 7 or the dispensing tube. The constriction valve is advantageously adapted to be a two-way valve for the flow of gas, and a one-way valve for the flow of paste. Thus, the constriction valve allows the flow of paste from the syringe into the dispensing tube, but paste is not allowed to passage the constriction in the opposite direction.

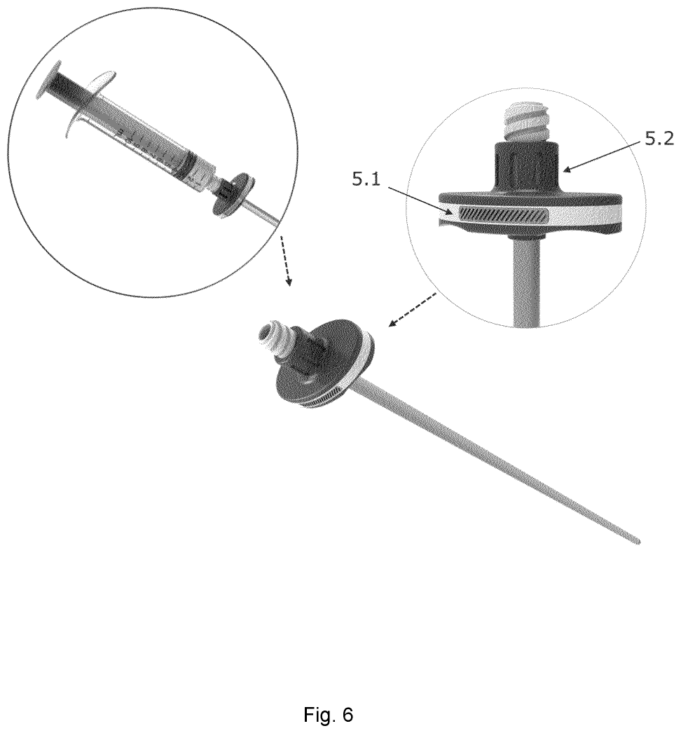

FIG. 6 shows a perspective view of an embodiment of the applicator tube, where the enhanced sections shown in circles, shows respectively the attachment to the syringe, and embodiments of the position of the valves within the valve system. The position of the first valve 5.1, exemplified as a one-way air valve, and the position of the second valve 5.2, exemplified as a one-way paste valve, is indicated. The second valve may be placed adjacent to the attachment to the syringe, as indicated in FIG. 6.

Kit of Parts

Advantageously, the applicator tube comprises a delivery tube and a valve system that are detachably attachable. Hence, the applicator tube may be stored and transported dissambled or as separate parts, in a compact and robust manner, and prior to use, the delivery tube and the valve system, optionally in the form of a transition unit, may be assembled to the applicator tube according to the present disclosure. Correspondingly after use, the kit may disassembled, and the parts may be separately disposed off and/or recycled.

An aspect of the disclosure relates to a kit of parts, comprising: a delivery tube, and a valve system, wherein the valve system is configured to be detachably attached to a proximal end of the delivery tube, and further configured for attachment to a syringe, and the valve system having a first configuration allowing aspiration of air from the surroundings, and a second configuration allowing expression of aspirated air into the delivery tube.

In an embodiment of the disclosure, the kit of parts comprises a delivery tube which is detachably attached to a valve system, and wherein optionally the valve system is in the form of a transition unit according to the present disclosure.

To ensure fast, easy, and reliable attachment and detachment between the delivery tube and the valve system or transition unit, the attachment is advantageously obtained by detachable fastening means, or detachably attached by a locking mechanism. Examples of detachable fastening means include a screw, click-on, slide-on, or snap-fit mechanism.

In an embodiment of the disclosure, the kit of parts comprises a valve system configured to be detachably attached to the delivery tube by a locking mechanism, such as a screw, click-on, or slide-on locking mechanism.

Paste

The applicator tube of the present disclosure is configured for dispensing paste, including emptying paste from an applicator tube, more specifically a medical paste. This means that the applicator tube is adapted for obtaining a high emptying or cleaning efficiency for paste. For residual medical paste remaining within the delivery tube, an emptying or cleaning efficiency of between 50-95% may be obtained, such as 80% efficiency. For example, it was observed that for a delivery tube containing 5 ml of residual paste, at least 4 ml of the residual paste was removed and discharged using the applicator tube and the associated method according to the present disclosure.

The efficiency of the presently disclosed applicator tube will depend on the paste properties. It was surprisingly found that the present applicator tube is especially efficient for medical paste. By the term “medical paste” is meant a paste comprising a bioactive agent. An example of a bioactive agent is thrombin.

A “bioactive agent” is defined as any agent, drug, compound, composition of matter or mixture which provides some pharmacologic, often beneficial, effect that can be demonstrated in vivo or in vitro. An agent is thus considered bioactive if it has interaction with or effect on a cell tissue in the human or animal body. As used herein, this term further includes any physiologically or pharmacologically active substance that produces a localized or systemic effect in an individual. Bioactive agents may be a protein, such as an enzyme. Further examples of bioactive agents include, but are not limited to, agents comprising or consisting of an oligosaccharide, a polysaccharide, an optionally glycosylated peptide, an optionally glycosylated polypeptide, an oligonucleotide, a polynucleotide, a lipid, a fatty acid, a fatty acid ester and secondary metabolites. It may be used either prophylactically, therapeutically, in connection with treatment of an individual, such as a human or any other animal. The term “bioactive agent” as used herein does not encompass cells, such as eukaryotic or prokaryotic cells.

A “paste” according to the present disclosure has a malleable, putty-like consistency, such as toothpaste. A paste is a thick fluid mixture of pulverized solid/solid in powder form with a liquid. A paste is a substance that behaves as a solid until a sufficiently large load or stress is applied, at which point it flows like a fluid, i.e. a paste is flowable. Flowables conform efficiently to irregular surfaces upon application. Pastes typically consist of a suspension of granular material in a background fluid. The individual grains are jammed together like sand on a beach, forming a disordered, glassy or amorphous structure, and giving pastes their solid-like character. It is this “jamming together” that gives pastes some of their most unusual properties; this causes a paste to demonstrate properties of fragile matter. A paste is not a gel/jelly. A “slurry” is a fluid mixture of a powdered/pulverized solid with a liquid, such as water. Slurries behave in some ways like thick fluids, flowing under gravity and being capable of being pumped if not too thick. A slurry may functionally be regarded as a thin, watery paste, but a slurry generally contains more water than a paste. Substantially water-insoluble powder particles, such as cross-linked gelatine particles, will form a paste upon mixing with an aqueous medium.

A “gel” is a solid, jelly-like material that can have properties ranging from soft and weak to hard and tough. Gels are defined as a substantially dilute cross-linked system, which exhibits no flow when in the steady-state. By weight, gels are mostly liquid, yet they behave like solids due to a three-dimensional cross-linked network within the liquid. It is the crosslinks within the fluid that give a gel its structure (hardness) and contribute to stickiness (tack). In this way gels are a dispersion of molecules of a liquid within a solid in which the solid is the continuous phase and the liquid is the discontinuous phase. A gel is not a paste or slurry. For example, non-crosslinked gelatine is soluble and forms a gel upon contact with an aqueous medium such as water.

For a medical paste to be discharged from a syringe and an applicator tube, it should be flowable, when subjected to a force applicable for a syringe. Thus, by the term “flowable paste” is meant a paste having a viscosity facilitating a steady flow, when subjected to a force applicable for a syringe. An example of a flowable paste is a paste having a viscosity between 500-3500 Pas, when measured at 30° C. and a relative humidity between 65-75%. In an embodiment of the disclosure, the paste is flowable.

Forming a medical paste, such as a flowable medical paste, requires mixing of the bioactive agent with a paste or a paste forming material. Typically, bioactive agents are stored in a solid and dried state, such as a powdered form, facilitating stable storage of the active agent, and flexible concentrations by mixing the bioactive agent with a diluent in an adjustable ratio. Thus, for the bioactive agent to be administered by a syringe injection, the solid bioactive agent must first be reconstituted. Forming a medical paste therefore typically requires the steps of mixing a solid bioactive agent with a liquid or diluent to reconstitute the bioactive agent, and subsequently mixing the reconstituted bioactive agent with a paste forming material, which may also be referred to as “paste precursor”.

By the term “paste forming material” is meant a material for forming a paste from a liquid phase, such as a reconstituted bioactive agent. Thus, a paste forming material may also be referred to as a precursor material for forming a paste.

The reconstituted bioactive agent is obtained by mixing the bioactive agent with a liquid with low viscosity, such as sterile water or saline water, thereby ensuring uniform reconstitution. Thus, the reconstituted bioactive agent is a liquid with low viscosity. A paste may be obtained from the reconstituted bioactive agent by adding a paste forming material, which inherently increases the viscosity.

### REFERENCE NUMBERS

1—Applicator tube
2—Delivery tube
2.1—Proximal end
2.2—Distal end
3— Transition unit/handle
4— Luer lock
5— Valve system
5.1— First valve
5.2— Second valve
6— Surroundings
7— First lumen
8— Second lumen
10— Paste
11— Gas
12— Insufflated body part
13—Trocar

### EXAMPLES

The invention is further described by the examples provided below.

### Example 1: Applicator Tube Containing a Low-Viscosity Paste

An applicator tube as illustrated in FIG. 3 was used, where the applicator tube was an endoscopic and/or laparoscopic applicator tube.

A syringe containing 10 ml of a low-viscosity paste was attached to the applicator tube. The low-viscosity paste was a medical paste having a particularly high flowability, corresponding to a viscosity around 500 Pas, when measured at 30° C. and a relative humidity between 65-75%.

The 10 ml of low-viscosity paste was expressed into the delivery tube, and 5 ml delivered to the target site. The delivery tube had an inner volume of 5 ml, and 5 ml of the low-viscosity paste therefore resided within the tube as residual paste due to the paste properties after the delivery.

Subsequently, the empty syringe was retracted, whereby air from the surroundings was aspirated into the syringe. The volume of aspirated air corresponded to the amount of low viscosity paste, i.e. 10 ml of air was aspirated. The air was subsequently injected into the delivery tube, and about 4 ml of the residual paste was expressed from the distal end of the delivery tube.

### Example 2: Applicator Tube Comprising a High-Viscosity Paste

An applicator tube as illustrated in FIG. 4 was used, where the applicator tube was an endoscopic and/or laparoscopic applicator tube.

A syringe containing 10 ml of a high-viscosity paste was attached to the applicator tube. The high-viscosity paste was a medical paste having a particularly low flowability, corresponding to a viscosity around 3500 Pas, when measured at 30° C. and a relative humidity between 65-75%.

The 10 ml of high-viscosity paste was expressed into the delivery tube, and 5 ml delivered to the target site. The delivery tube had an inner volume of 5 ml, and 5 ml of the high-viscosity paste therefore resided within the tube as residual paste due to the paste properties after the delivery.

Subsequently, the empty syringe was retracted, whereby air from the surroundings was aspirated into the syringe. Due to the low flowability and high viscosity of the paste, no paste was aspirated into the syringe. The volume of aspirated air corresponded to the amount of low viscosity paste, i.e. 10 ml of air was aspirated. The air was subsequently injected into the delivery tube, and about 4 ml of the residual paste was expressed from the distal end of the delivery tube.

### Example 3: Applicator Tube Used within an Insufflated Body Part

An applicator tube comprising a comprising a combination valve system, as the duckbill/umbrella combination valve illustrated in FIG. 9 was used, where the applicator tube was an endoscopic and/or laparoscopic applicator tube inserted within an insufflated body part.

A syringe containing 10 ml of a low-viscosity paste was attached to the applicator tube.

The low-viscosity paste was a medical paste having a particularly high flowability, corresponding to a viscosity around 500 Pas, when measured at 30° C. and a relative humidity between 65-75%.

The 10 ml of low-viscosity paste was expressed into the delivery tube, and 5 ml delivered to the target site. The delivery tube had an inner volume of 5 ml, and 5 ml of the low-viscosity paste therefore resided within the tube as residual paste due to the paste properties after the delivery.

Subsequently, the empty syringe was retracted, whereby insufflated gas from the surroundings of the inserted delivery tube was aspirated into the syringe, as illustrated in FIG. 7B. The volume of aspirated gas corresponded to the amount of low viscosity paste, i.e. 10 ml of insufflated gas was aspirated. The gas was subsequently injected into the delivery tube, and about 4 ml of the residual paste was expressed from the distal end of the delivery tube.

Items

The presently disclosed may be described in further detail with reference to the following items.
1. An endoscopic and/or laparoscopic applicator tube for delivering a paste from a syringe, comprising:
   a delivery tube comprising a distal end for delivering the paste,
   a valve system attached to a proximal end of the delivery tube and configured for attachment to the syringe, the valve system having a first configuration allowing aspiration of gas from the tube surroundings, and a second configuration allowing expression of aspirated gas into the delivery tube,
   such that the applicator tube is configured for transporting a volume of aspirated gas into and through the delivery tube when an attached syringe is aspirated and expressed.
2. An applicator tube for delivering a paste from a syringe, comprising:
   a delivery tube comprising a distal end for delivering the paste,
   a valve system attached to a proximal end of the delivery tube and configured for attachment to the syringe, the valve system having a first configuration allowing aspiration of gas from the tube surroundings, and a second configuration allowing expression of aspirated gas into the delivery tube,
   such that the applicator tube is configured for transporting a volume of aspirated gas into and through the delivery tube when an attached syringe is aspirated and expressed.
3. The applicator tube according to item 2, wherein the applicator tube is an endoscopic and/or laparoscopic applicator tube.
4. The applicator tube according to any of the preceding items, wherein the applicator tube is adapted for insertion into a trocar.
5. The applicator tube according to any of the preceding items, wherein the delivery tube has a length between 20-150 cm, more preferably between 25-80 cm, such as between 30-60 cm.
6. The applicator tube according to any of the preceding items, wherein the delivery tube comprises an internal diameter of between 2-15 mm, more preferably between 3-8 mm, such as between 4-6 mm or 3-5 mm.
7. The applicator tube according to any of the preceding items, wherein the delivery tube has a stiffness of above 0.5, 1.5 or 2 GPa, more preferably above 50 or 60 GPa.
8. The applicator tube according to any of the preceding items, wherein the delivery tube contains a volume of between 3-20 ml, preferably between 4 −10 ml, such as 5 ml.
9. The applicator tube according to any of the preceding items, wherein the delivery tube comprises a material selected from the group of: metals, plastics, polymers, glass, glass fibers, carbon fibers, polymer fibers, composites such as fiber-reinforced materials, and combinations thereof.
10. The applicator tube according to any of the preceding items, further comprising a transition unit attached to the proximal end of the delivery tube, wherein preferably the transition unit is adapted as a handle.
11. The applicator tube according to any of the preceding items, further comprising a Luer lock for attaching a syringe.
12. The applicator tube according to any of the preceding items, wherein the delivery tube comprises or is a cannula.
13. The applicator tube according to any of the preceding items, comprising a formable distal section configured to be shaped into a desired configuration.
14. The applicator tube according to item 13, wherein the formable distal section comprises a malleable member configured to maintain a configuration after a deformation, wherein said malleable member optionally is a malleable wire or a malleable mesh.
15. The applicator tube according to item 14, wherein the malleable member is located on the inside, outside, or within the delivery tube wall.
16. The applicator tube according to any of the preceding items, wherein the valve system comprises at least two valves, or a two valve function combination valve, such as a duckbill/umbrella combination valve.
17. The applicator tube according to item 16, wherein the valve system comprises at least a first one-way valve, and/or a first constriction valve.
18. The applicator tube according to any of items 16-17, wherein the valve system comprises at least two one-way valves.
19. The applicator tube according to any of items 16-18, wherein the valve system comprises a constriction valve with a cross sectional area of between 20-90%, more preferably between 30-80%, and most preferably between 40-60% of the cross sectional area of the delivery tube.
20. The applicator tube according to any of items 10-19, wherein the valve system is integrated into the transition unit and/or the proximal end of the delivery tube.
21. The applicator tube according to any of items 10-20, wherein the valve system or the transition unit comprises a first lumen having a first proximal opening and a first distal opening, wherein the first distal opening is in fluid communication with the delivery tube, and at least one second lumen having a second proximal opening and a second distal opening, wherein the second distal opening is in fluid communication with the tube surroundings.
22. The applicator tube according to item 21, wherein the first proximal opening and the second proximal opening are the same.
23. The applicator tube according to any of items 21-22, wherein the second distal opening is located within a proximal end of the delivery tube, such as within the transition unit.
24. The applicator tube according to item 23, wherein the extension of the second lumen is oriented at an angle from the extension of the first lumen, such as extending perpendicular to the first lumen.
25. The applicator tube according to any of items 23-24, wherein the second distal opening is in fluid communication with a gas container.
26. The applicator tube according to any of items 21-22, wherein the second distal opening is located within a distal end of the delivery tube, optionally at a distance below 2, 5, 6, 7, 8, 10, or 15 cm from the distal end of the delivery tube.
27. The applicator tube according to item 26, wherein the second distal opening comprises one or more apertures located at the delivery tube outside wall and/or at the delivery tube wall flange.
28. The applicator tube according to any of items 26-27, wherein the extension of the second lumen is in parallel to the extension of the first lumen.
29. The applicator tube according to any of items 26-28, wherein the second lumen is concentric with the first lumen.
30. The applicator tube according to any of items 26-29, wherein the second lumen is located on the inside, outside, or within the delivery tube wall.
31. The applicator tube according to any of items 21-30, comprising multiple second lumens, such as 2, 4, 6, 8, or 10 second lumens.
32. The applicator tube according to any of items 21-31, wherein the valve system comprises at least a first one-way valve, and preferably wherein the first one-way valve is placed within the second lumen.
33. The applicator tube according to any of items 21-32, wherein the first lumen comprises a part with a reduced cross section area.
34. The applicator tube according to item 33, wherein the first lumen comprises a part with a reduced cross sectional are of between between 20-90%, more preferably between 30-80%, and most preferably between 40-60% of the cross sectional area of the lumen.
35. The applicator tube according to any of the preceding items, wherein the valve system comprises a second one-way valve.
36. The applicator tube according to item 35, wherein the second one-way valve is placed within the first lumen.
37. A method of emptying an applicator tube, comprising the steps of:
   a) providing the applicator tube according to any of items 1-36,
   b) attaching a syringe to the delivery tube,
   c) aspirating gas into the syringe,
   d) expressing the gas from the syringe into the delivery tube,
   whereby the applicator tube is emptied.
38. The method according to item 37, wherein the attached syringe in (b) contains paste, and further comprising the step of expressing at least a part of the paste from the syringe into the delivery tube before aspirating air in (c).
39. The method according to any of items 37-38, wherein the gas is an insufflation gas selected from group of: air, CO₂, nitrous oxide (N₂O), helium (He), and combinations thereof.
40. A kit of parts, comprising: a delivery tube, and a valve system, wherein the valve system is configured to be detachably attached to a proximal end of the delivery tube, and further configured for attachment to a syringe, and the valve system having a first configuration allowing aspiration of gas from the surroundings, and a second configuration allowing expression of aspirated gas into the delivery tube.
41. The kit of parts according to item 40, wherein the valve system is configured to be detachably attached to the delivery tube by a locking mechanism, such as a screw, click-on, or slide-on locking mechanism.

### REFERENCES

[1] US 2018 303531

1. An endoscopic and/or laparoscopic applicator tube for delivering a paste from a syringe, comprising:
a delivery tube comprising a distal end for delivering the paste,
a valve system attached to a proximal end of the delivery tube and configured for attachment to the syringe, the valve system having a first configuration allowing aspiration of gas from the tube surroundings, and a second configuration allowing expression of aspirated gas into the delivery tube,
such that the applicator tube is configured for transporting a volume of aspirated gas into and through the delivery tube when an attached syringe is aspirated and expressed.

2. The applicator tube according to claim 1, wherein the delivery tube has a length between 20-150 cm, and/or wherein the delivery tube contains a volume of between 3-20 ml.

3. The applicator tube according to claim 1, further comprising a transition unit attached to the proximal end of the delivery tube.

4. The applicator tube according to claim 1, wherein the valve system comprises at least two valves, or a two valve function combination valve, such as a duckbill/umbrella combination valve.

5. The applicator tube according to claim 4, wherein the valve system comprises at least a first one-way valve.

6. The applicator tube according to claim 4, wherein the valve system comprises at least two one-way valves.

7. The applicator tube according to any of claim 4, wherein the valve system comprises a constriction valve with a cross sectional area of between 20-90% of the cross sectional area of the delivery tube.

8. The applicator tube according to claim 1, wherein the valve system is integrated into the transition unit, and/or the proximal end of the delivery tube.

9. The applicator tube according to claim 1, wherein the valve system or the transition unit comprises a first lumen having a first proximal opening and a first distal opening, wherein the first distal opening is in fluid communication with the delivery tube, and a second lumen having a second proximal opening and a second distal opening, wherein the second distal opening is in fluid communication with the surroundings.

10. The applicator tube according to claim 9, wherein the valve system comprises at least a first one-way valve.

11. The applicator tube according to claim 9, wherein the first lumen comprises a part with a reduced cross sectional area.

12. The applicator tube according to claim 9, wherein the valve system comprises a second one-way valve.

13. A method of emptying an applicator tube, comprising:
a) providing the applicator tube according to claim 1,
b) attaching a syringe to the delivery tube,
c) aspirating gas into the syringe,
d) expressing the gas from the syringe into the delivery tube,
whereby the applicator tube is emptied.

14. The method according to claim 13, wherein the gas is an insufflation gas selected from group of: air, CO₂, nitrous oxide (N₂O), helium (He), and combinations thereof.

15. A kit of parts for the endoscopic and/or laparoscopic applicator tube according to claim 1, comprising:
the delivery tube, and
the valve system,
wherein the valve system is configured to be detachably attached to a proximal end of the delivery tube, and further configured for attachment to a syringe, the valve system having a first configuration allowing aspiration of air from the surroundings, and a second configuration allowing expression of aspirated air into the delivery tube.

16. The applicator tube according to claim 1, wherein the valve system comprises a duckbill/umbrella combination valve.

17. The applicator tube according to claim 9, wherein the first proximal opening and the second proximal opening are the same, and configured to be fluid communication with the syringe.

18. The applicator tube according to claim 10, wherein the first one-way valve is placed within the second lumen.

19. The applicator tube according to claim 12, wherein the second one-way valve is placed within the first lumen.

20. The method according to claim 13, wherein the attached syringe in (b) contains paste, and the method further comprising expressing at least a part of the paste from the syringe into the delivery tube before aspirating gas in (c).
